(19) United States
(12) Patent Application Publication
(10) Pub. No.: US 2016/0287829 A1
(43) Pub. Date: Oct. 6, 2016

(54) Cushioned Sleep Apnea Mask

(71) Applicants: Tom Whitehead, Edinburg, IL (US); Tina Whitehead, Edinburg, IL (US)

(72) Inventors: Tom Whitehead, Edinburg, IL (US); Tina Whitehead, Edinburg, IL (US)

(21) Appl. No.: 15/006,330

(22) Filed: Jan. 26, 2016

Related U.S. Application Data
(60) Provisional application No. 62/140,574, filed on Mar. 31, 2015.

(51) Int. Cl. A61M 16/06; A61M 16/00

(52) U.S. Cl. CPC A61M 16/0622; A61M 16/0683; A61M 16/0057

(57) ABSTRACT

A cushioned sleep apnea mask to provide comfort to the user while wearing the sleep apnea mask. The cushioned sleep apnea mask provides a body that can be secured around the user's head so as to cushion portions of the user's head contacted thereby. The sleep apnea mask includes a rear portion to be positioned on the rear of the user's head, wherein a pad is disposed on the interior surface thereof. An upper strap extends around the user's forehead and a lower strap extends around the user's face. The front of the body includes a breathing device that can be placed over the user's nose, wherein the breathing device is connected to the upper strap and lower strap of the sleep apnea mask.

### CROSS REFERENCE TO RELATED APPLICATION

This application claims the benefit of U.S. Provisional Application No. 62/140,574 filed on Mar. 31, 2015. The above identified patent application is herein incorporated by reference in its entirety to provide continuity of disclosure.

### BACKGROUND OF THE INVENTION

### Field of the Invention

The present invention relates to sleep apnea masks. More specifically, the present invention provides a cushioned sleep apnea mask adapted to alleviate discomfort that is often experienced when wearing a traditional sleep apnea mask. The sleep apnea mask comprises a body having a rear portion to be disposed on the back of the user's head, wherein an upper strap and lower strap extend around the user's forehead and face, respectively. The rear portion comprises a pad thereon so as to provide comfort to the back of the user's head by preventing the straps from causing discomfort to the user.

Many people wear sleep apnea masks while sleeping in order to correct problems related to sleep apnea. By wearing a sleep apnea mask, people suffering from sleep apnea are able to obtain a full night's rest without periodically waking up throughout the night. Sleep apnea masks support a breathing mask over a person's nose so as to keep the person's airways open while they sleep. However, conventional sleep apnea masks include straps that extend around a user's head and are uncomfortable to wear. The straps may press into the user's head and face, causing discomfort, which makes it more difficult for the person to obtain a full night's rest. Further, the straps may pull on the user's hair, causing additional discomfort or pain.

Devices have been disclosed in the prior art that relate to cushioned headgear. These include devices that have been patented and published in patent application publications. These devices generally relate to pillows and cushions, such as U.S. Pat. No. 6,651,256, U.S. Pat. No. 941,043, and U.S. Published Patent Application Number 2013/0232693. Other patents provide headgear cushions, such as U.S. Published Patent Application Number 2010/0018535 and U.S. Pat. No. 8,443,805.

These prior art devices have several known drawbacks. These devices fail to provide comfort for the rear of a user's head while wearing a sleep apnea mask or other similar headgear. Further, such devices fail to provide cushioning to prevent the straps or body of the headgear from causing discomfort to the user when the user is wearing the headgear.

In light of the devices disclosed in the prior art, it is submitted that the present invention substantially diverges in design elements from the prior art and consequently it is clear that there is a need in the art for an improvement to existing sleep apnea masks. In this regard the instant invention substantially fulfills these needs.

### SUMMARY OF THE INVENTION

In view of the foregoing disadvantages inherent in the known types of sleep apnea masks now present in the prior art, the present invention provides a new sleep apnea mask wherein the same can be utilized for providing convenience for the user when sleeping while wearing a sleep apnea mask.

It is therefore an object of the present invention to provide a new and improved sleep apnea mask comprising a body adapted to be worn on a user's head that includes a rear portion that rests on the back of the user's head, an upper strap that extends around the forehead of the user, and a lower strap that extends around the face of the user beneath the nose.

It is another object of the present invention to provide a sleep apnea mask composed of a flexible material that can stretch so that the sleep apnea mask fits any of various users.

Another object of the present invention is to provide a sleep apnea mask comprising a breathing device disposed on a front portion thereof adapted to be placed over the nose of the user.

Yet another object of the present invention is to provide a sleep apnea mask that does not obstruct the user's vision or hearing.

Another object of the present invention is to provide a sleep apnea mask that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of the sleep apnea mask.

FIG. 2 shows a side perspective view of the sleep apnea mask.

FIG. 3 shows a side view of the sleep apnea mask as worn by a user.

### DETAILED DESCRIPTION OF THE INVENTION

Reference is made herein to the attached drawings. Like reference numerals are used throughout the drawings to depict like or similar elements of the sleep apnea mask. For the purposes of presenting a brief and clear description of the present invention, the preferred embodiment will be discussed as used for alleviating discomfort associated with wearing conventional sleep apnea masks. The figures are intended for representative purposes only and should not be considered to be limiting in any respect.

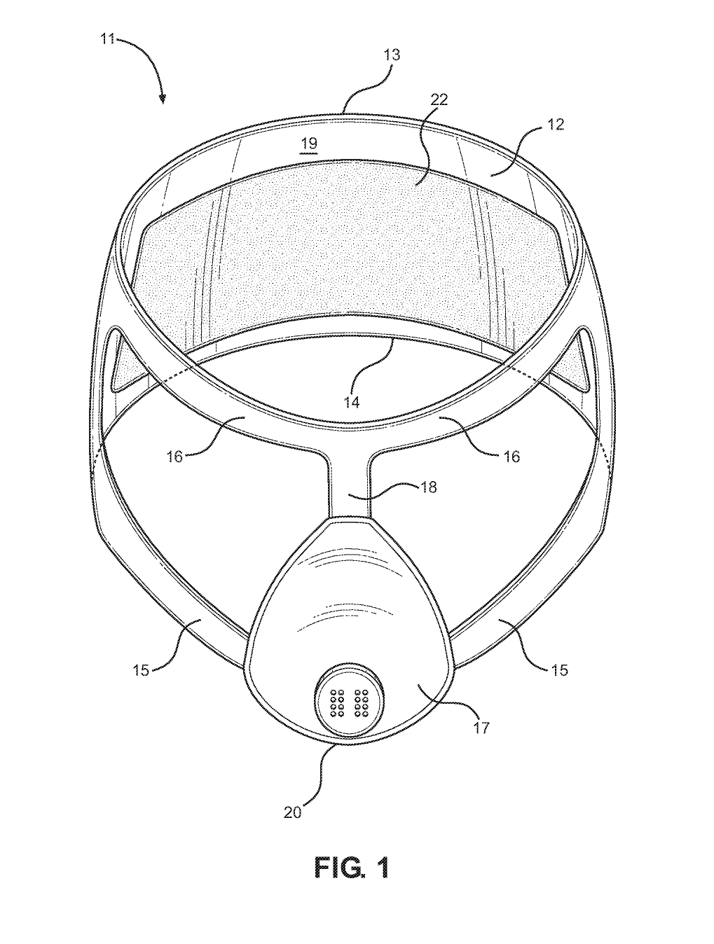

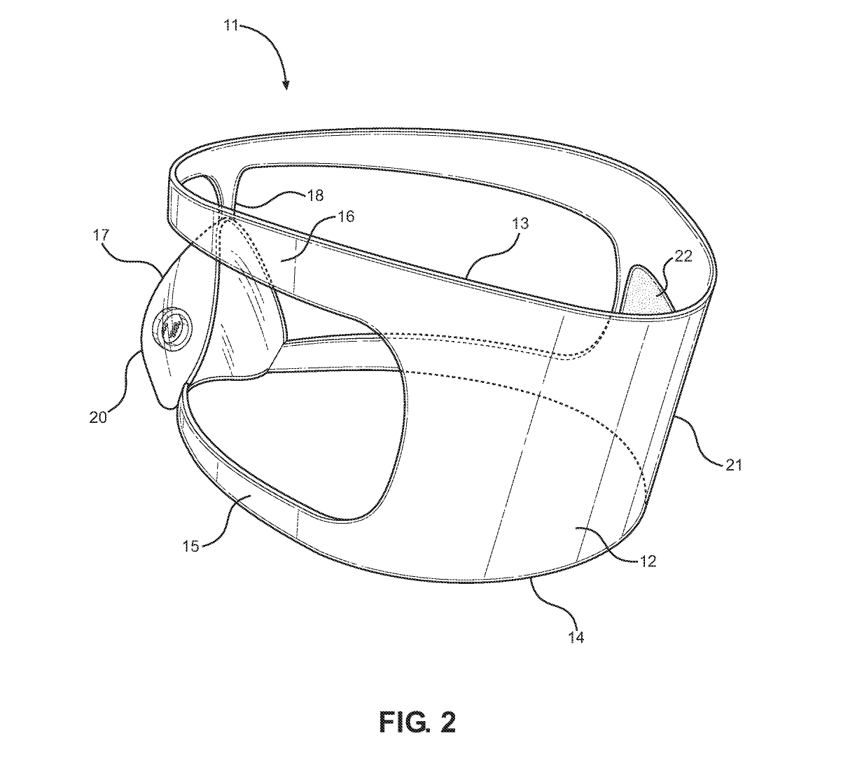

Referring now to FIGS. 1 and 2, there is shown a front perspective view of the sleep apnea mask and a side perspective view of the sleep apnea mask, respectively. The sleep apnea mask 11 comprises a body 12 adapted to be worn around the head of the user. The sleep apnea mask 11 is preferably composed of a flexible material so that it can stretch to snugly fit onto the head of a user, and so that the sleep apnea mask 11 can contour to the shape of the user's head. Further, the flexible material allows the sleep apnea mask 11 to be worn by any of various users. The sleep apnea mask 11 is further composed of a padded material to provide comfort to the user and to prevent the sleep apnea mask from contacting the user's skin.

The body 12 comprises a rear portion 21 adapted to be positioned on the back of a user's head. The interior surface 19 of the rear portion 21 comprises a pad 22 thereon. The pad 22 is positioned so as to relieve discomfort associated with the rear portion 21 pressing against the back of the user's head. The pad 22 is permanently affixed to the rear portion 21 of the body 12, and may be secured via adhesives or stitching. The pad 22 is preferably substantially rectangular in configuration. Further, the pad 22 is composed of a foam material. However, in alternate embodiments, the pad 22 is composed of alternate padded or cushioning materials, such as cotton, among others.

An upper strap 16 extends from the rear portion 21 so as to wrap around the user's forehead. In this way, the upper strap 16 encircles the user's head. Thus, the upper strap 16 forms a loop around the user's head in order to secure the sleep apnea mask 11 in position on the user's head. Similarly, a lower strap 15 extends from the rear portion 21 so as to extend around the user's face, preferably in an area beneath the user's nose and above the user's lips. The upper strap 16 and lower strap 15 are substantially parallel to one another and are separated by a space. In this way, the sleep apnea mask 11 does not cover the user's ears or eyes in order to prevent the sleep apnea mask 11 from obstructing the user's vision or hearing.

In some embodiments, the pad 22 on the rear portion of the body 12 extends along the interior surface of the upper strap 16 and/or lower strap 15. The pad 22 helps to prevent the straps 15, 16 from irritating the skin of the user or pulling on the user's hair. In some embodiments, the interior surface of the body 12 is substantially covered by the pad 22.

The front portion 20 of the sleep apnea mask 11 comprises a breathing device 17 thereon. The breathing device 17 comprises a substantially triangular configuration and is adapted to be placed over the user's nose. The breathing device 17 is preferably a continuous positive airway pressure (CPAP) mask adapted for connection to a CPAP machine, however, in alternate embodiments other similar types of breathing devices 17 can be used. The breathing device 17 is disposed between and is connected to the lower strap 15 and upper strap 16. In the illustrated embodiment, the breathing device 17 is connected to the upper strap 16 via a connecting strap 18 that extends along the bridge of the user's nose. However, in alternate embodiments, the breathing device 17 is directly connected to the upper strap 16. In this way, the breathing device 17 is supported in a fixed position between the lower strap 15 and upper strap 16.

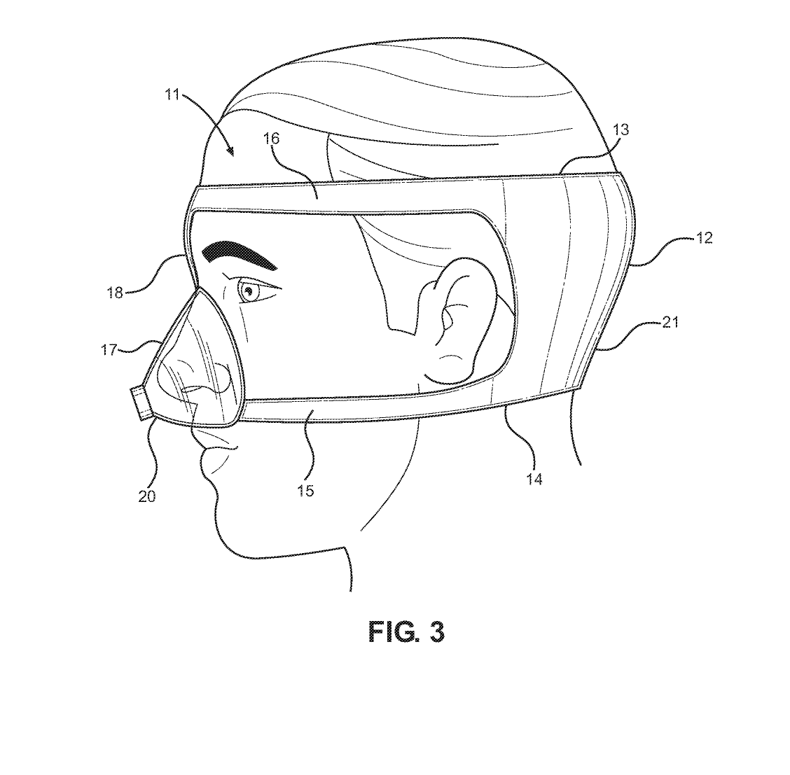

Referring now to FIG. 3, there is shown a side view of the sleep apnea mask as worn by a user. In operation, a user can position the sleep apnea mask 11 on his or her head. The user can stretch the body 12 of the sleep apnea mask 11 to fit over his or her head, wherein the sleep apnea mask 11 will fit snugly onto the user's head and will contour to the shape of the user's head. The rear portion 21 of the sleep apnea mask 11 is positioned on the back of the user's head. The interior surface of the rear portion 21 includes padding thereon to provide comfort to the user. An upper strap 16 extends around the user's forehead from the upper end 13 of the body 12. Similarly, a lower strap 15 extends around the user's face, preferably below the user's nose and above the user's lips, wherein the lower strap 15 extends from the lower end 14 of the body 12. In this way, the sleep apnea mask 11 provides cushioning in areas where sleep apnea masks commonly contacts a user's head or face and may cause discomfort. The sleep apnea mask 11 prevents the strap thereof from directly contacting the user's skin and helps to prevent the sleep apnea mask 11 from rubbing against or pressing into the user's head or face.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A sleep apnea mask, comprising:
a body having a rear portion adapted to be positioned on a back of a head of a user, wherein the rear portion includes a pad on an interior surface thereof;
an upper strap extending from said rear portion and adapted to encircle said head of said user in an area corresponding to a forehead of said user;
a lower strap extending from said rear portion and adapted to encircle said head of said user in an area corresponding to a lower portion of said head of said user, such that said upper strap and said lower strap are spaced from one another;
a breathing device connected to said lower strap and adapted to be positioned over a nose of the user.

2) The sleep apnea mask of claim 1, wherein the body comprises a flexible material adapted to contour to the head of the user.

3) The sleep apnea mask of claim 1, wherein the breathing device is connected to the lower strap at a front portion of the body.

4) The sleep apnea mask of claim 3, wherein the breathing device is further connected to the upper strap.

5) The sleep apnea mask of claim 1, wherein the pad on the interior surface of the rear portion of the body extends along an interior surface of the upper strap.

6) The sleep apnea mask of claim 1, wherein the pad is composed of a foam material.

7) The sleep apnea mask of claim 1, wherein the breathing device comprises a CPAP mask.
